(19) United States
(12) Patent Application Publication
(10) Pub. No.: US 2020/0038252 A1
(43) Pub. Date: Feb. 6, 2020

(54) TRI-LAYERED WOUND DRESSING AND METHOD THEREFOR

(71) Applicant: Joseph Spiro, Flushing, NY (US)

(72) Inventor: Joseph Spiro, Flushing, NY (US)

(21) Appl. No.: 16/050,226

(22) Filed: Jul. 31, 2018

(51) Int. Cl. A61F 13/02; A61F 13/00

(52) U.S. Cl. CPC A61F 13/0243; A61F 13/0206; A61F 2013/00319; A61F 2013/00272; A61F 2013/00182; A61F 13/00059

(57) ABSTRACT

A multi-layer wound dressing includes a base layer with a circumscribing web of material defining a central opening of sufficient size to expose a wound area. An intermediate layer is the bandage layer that is placed over the opening in the base layer. An outer cover layer located above and over the bandage layer makes a sealing connection with the base layer and totally covers the intermediate bandage layer, providing sealing for the wound area and easy and repeated replacement of the bandage layer.

### BACKGROUND OF THE INVENTION

The present invention is generally directed to wound dressings and, more particularly, to a three-layer or tri-component wound dressing that provides improved sealing of the wound site and easier and less painful re-dressing of wounds.

Traditional methods and products for changing wound bandages and dressings are time-consuming and often accompanied by pain. Children and sometime adults resist proper care-taking of wounds for fear of pain. Also, often time wounds are cautioned from becoming wet, making for inconvenient bathing or showering options.

Wound dressings are generally employed to provide an outer physical layer of protection for a wound such as a burned area of skin or an open or closed wound resulting from injury or surgical procedures and the like. Conventional approaches to protecting a wound typically involve the application of a wound dressing formed with an adhesive layer applied to a plastic strip that includes a patch of a soft and absorbent wound-curing component such as a gauze or cloth material, sometime impregnated with medicinal substances. These products have a tendency to form a bond over time with the wound, making the process of exchanging a wound dressing painful and also susceptible to disturbing the partially healed wound site, which prolongs the wound healing time.

Many, particularly children, therefore resist and fail to attend to the needed frequent cleansing of the wound and the adjacent skin area. Even with adults, the need to cleanse a wound and then reapply a wound dressing is not easily performed when the wound is located on an arm or a hand of a person and that person only has one hand to attend to the dressing of the wound.

Various wound dressings are described in U.S. Pat. Nos. 5,106,629, 3,645,835 and D745,973, and also in United States Patent Application Publication Nos. US 2014/0358058 and US 2012/0238932, the contents of which issued United States Patents and published United States Patent Applications are incorporated by reference herein.

### SUMMARY OF THE INVENTION

Accordingly, it is a general object of the present invention to provide wound dressings and bandages and methods that overcome the above-mentioned drawbacks of the prior art.

It is another object of the present invention to provide wound-dressings that are easily applicable to wounds and that enable easy removal of the wound dressing, cleansing of the wound site and re-dressing of the wound.

It is another object of the invention to provide wound dressings with improved sealing of the wound site against wetting from the outside and/or against body liquids oozing through the dressing.

It is yet another object of the invention to provide wound dressings that provide in certain applications greater protection against accidental painful contact with the wound area.

The foregoing and other objects of the invention are realized with a multi-layer wound dressing, comprising: a base layer comprising a circumscribing web of material defining a central opening of a pre-determined size to expose a wound area, the web having an adhering bottom surface for being adhered to and contact a region around the wound area; an intermediate layer comprising a bandage layer, located over said opening of said base layer, the bandage layer being configured to be applied against the wound area; and an outer, cover layer located over said bandage layer, said cover layer including a cover connection structure configured to make a sealed connection to said base layer, enabling repeated disconnection and reconnection of said cover layer to said base layer and repeated replacement of said bandage layer. The cover connection structure may comprise a tongue and groove connection, and the tongue and groove connection may comprise a tongue structure on the cover layer and a circumscribing groove on the base layer. The cover connection structure may comprise an adhesive connection between the base layer and the cover layer. The bandage layer may also feature connection structure configured to connect an edge of the bandage layer to the base layer. Preferably, the cover connection structure is located more peripherally out than the bandage connection structure, and the bandage layer may comprise gauze material. The outer shape of said base layer can be any one of oblong, oval, square, rectangular, heart-shaped, and the like.

Preferably, the multi-layer wound dressing includes orientation marks that enable the cover layer to be rotationally oriented relative to the base layer, and the orientation marks provided on both the base layer and on the cover layer. The wound dressing preferably includes cover layer tabs that are configured to be hand-graspable and constructed to allow lifting of the cover layer off the base layer, and lifting tabs projecting from outer edge of the base layer for enabling grasping of the cover layer and removal of the same from a person's skin. Also, the bandage layer preferably includes a central and removable wound-site bandage that is surrounded by a more rigid circumscribing material, and preferably the bandage layer is formed of a plurality of overlapping webs of gauze material, preferably at least three webs of gauze material that are peelable from each other, and wherein the three layers include a lowest layer that comprises gauze material of a lowest weave density and a top-most layer that has a highest gauze weave density.

The cover layer is preferably made from a semi-rigid material that resists bending, to prevent a force applied to it from the outside from being transmitted to the wound site, and include a transparent window. In an embodiment, the cover layer includes an outer cover with a plurality of perforations therein, the perforations having opening sizes small enough to substantially prevent outside moisture or wetness from entering the wound site and large enough to allow pressurized air or liquid underneath said cover layer to be vented to the atmosphere.

Other features and advantages of the present invention will become apparent from the following description of the invention which refers to the accompanying drawings.

### BRIEF DESCRIPTION OF THE DRAWINGS

FIG. 1 is an exploded perspective of the three-layer wound dressing of the present invention.

FIG. 2 shows the wound dressing of FIG. 1 in an assembled form.

FIGS. 3a, 3b and 3c show the three components of the wound dressing diagrammatically.

FIGS. 4a, 4b, 4c and 4d show differently shaped wound dressings that utilize the dressing concepts of the present invention.

FIG. 5 shows the intermediate layer of the wound dressing invention formed internally of three layers.

FIGS. 5a, 5b and 5c illustrate intermediate wound dressing of FIG. 5.

Other features and advantages of the present invention will become apparent from the following description of the invention which refers to the

### DETAILED DESCRIPTION OF EMBODIMENTS OF THE INVENTION

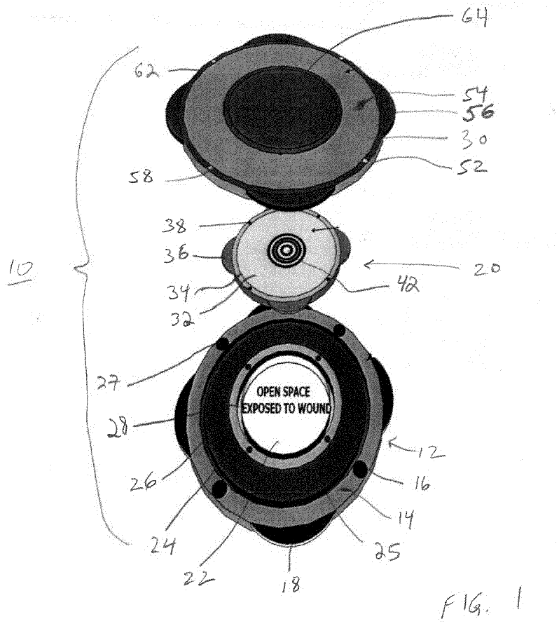

Referring to the drawings, in general, the invention can be described as a three-tier bandage or gauze replacement and waterproofing system 10, the three main components of which are depicted in FIG. 1. Thus, the bandaging system 10 includes a base layer 12, a bandage layer 20 and a waterproofing layer 30. Typically, to dress a wound, for example a skin cut or the like, one first applies the base layer 10 around the wound in such manner that the wound is not touched by the base layer 12 and the wound site becomes centrally located in the open space 22 at the center of the base layer 12. The base layer 12 is attached only once. The base layer or component 12 contains pre-formed grooves or other attachment means that enable the second layer, i.e. the bandage layer 20 and finally the protective layer 50 to be attached to the base layer 12.

More specifically, the base layer 12 has a circumscribing region 14 extending around the open space 22, defining a surrounding groove 24 that extends between an outer peripheral region 14 and the central body portion 26. The outer groove 24 provides a sealing connection to the cover layer 30, as explained further on. Optionally, the base layer 12 also defines an inner circumscribing connection groove 28 for effecting a sealing connection to the bandage layer 20. The opposite, rear (non-depicted) side of the base layer 12 has a conventional, adhesive that is typically covered by a removable sheet that is peeled away to allow the base layer 12 to be adhesively attached to the skin around the wound.

The bandage layer 20 has a body 32 with an optional projecting tongue 34 and tabs 36. In use, the bandage layer 20 is applied directly against the wound site and its tongue 34 fits into the groove 28 on the base layer 12, providing assurance that the bandage layer will not move about, as well as a measure of sealing for the wound site. The center portion 42 can be made replaceable and comprise a centralized gauze that can be removed and then replaced in accordance with an option of the present invention. Once the gauze has been located over the wound, the system 10 is fully sealed with the cover layer 30 being applied such that its circumscribing tongue 62, which extends between the outer edge and the inner body 54 is fitted in the groove 28 on the base layer 12. Thus, the outer, waterproof cover 30 can be easily attached to the system 10 by having its tongue 62 fitted into the groove 28 in the base layer, making it simple and convenient for the patient to take a shower or bath and not get moisture into the wound.

After the wound has healed, the base layer 12 is removed like with any bandage. The discomfort and pain of removing the dressing is thus only required one time.

As the wound is healing and it is desirable to exchange the bandage layer 20, it is easy to grasp the outer cover 30 by its tabs 56, lift it off the wound and thereafter remove and replace the bandage layer 20 entirely, or if desired replace only the gauze component 42 at the center thereof. The alignment marks 27 on the base 12 and the similar alignment marks 58 on the cover layer 30, allow these components to align with one another, so that the tabs 18 and 56 are peripherally offset and easily distinguishable. Thus, it is easy to grasp the correct tab when wishing to lift either the outer cover layer 30 and later (and ultimately) the base layer 12.

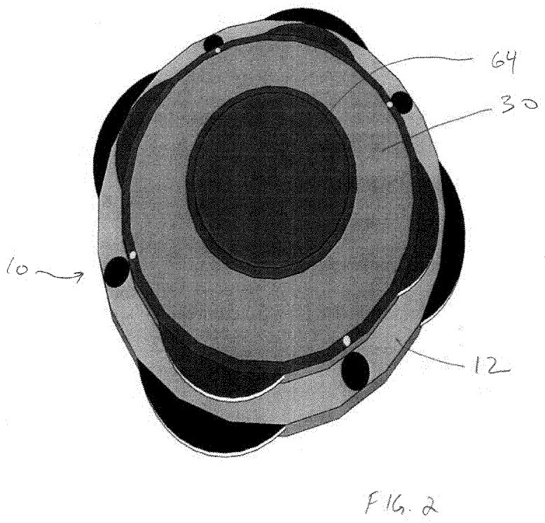

It is also beneficial to view FIGS. 3a, 3b, and 3c which diagrammatically illustrate the base layer 12′, the bandage layer 20′ and the cover layer 30′ in slightly modified form. In these drawings, it is easier to identify the pair of circumscribing, outer and inner grooves 24 and 28 on the base layer 12, as well as the projecting tongues 62 and 34 on the cover layer 30 and on the bandage 20, which fit into the circumscribing grooves 24, 28 on the base layer 12.

The center area 64 of the cover 30 is separately identified because it can consist either of just an opaque unitary extension of the overall cover 30, or it might consist of a transparent material, forming a sealed window made of a plastic material that allows examining the condition of the wound dressing 20 underneath. One option of the invention is to make that window somewhat rigid so that if somebody deliberately or accidentally applies pressure to the wound area, that pressure is not communicated to the bandage below and does not disturb the healing of the wound or cause any physical pain.

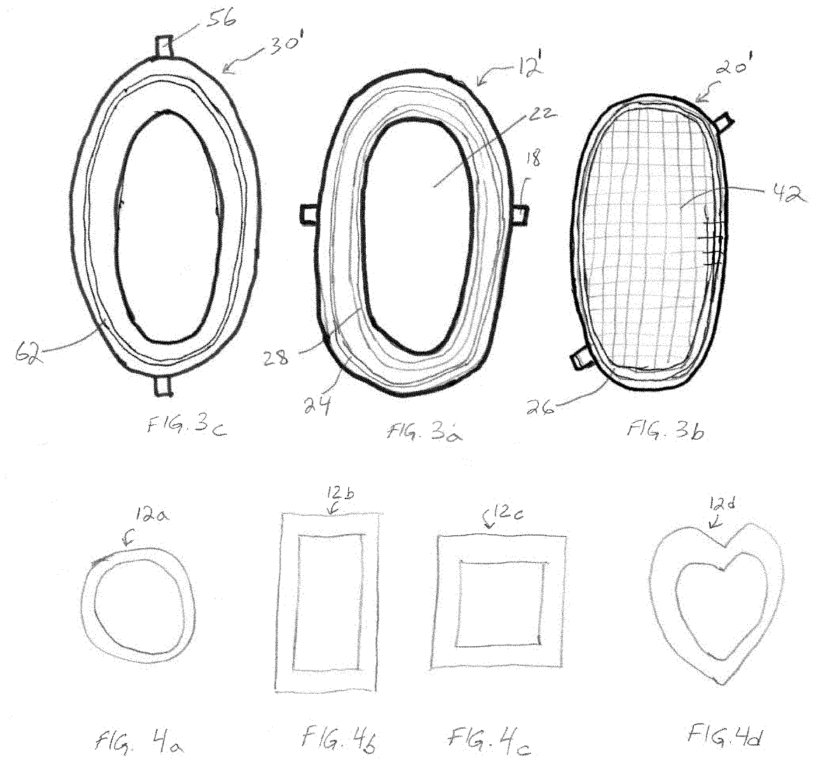

Although the invention has been described above with reference to a generally oval or somewhat elongated wound dressing, the invention can be easily implemented in many different shapes without loss of functionality, as illustrated in FIGS. 4a, 4b, 4c and 4d. These drawing figures respectively show a circular, a rectangular, a square and a heart-shaped dressing. However, any and all other shapes are capable of implementing the three-part bandaging system of the present invention.

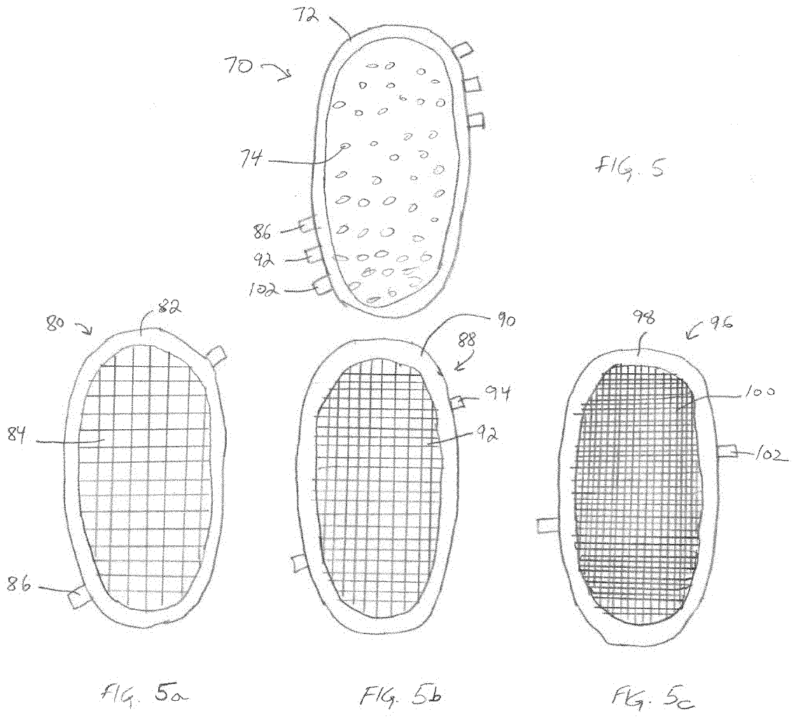

In accordance with a further development of the present invention, reference is made to FIG. 5 and the related FIGS. 5a, 5b and 5c that show an improved bandage layer 70 in a configuration whereby the bandage is itself fabricated of three layers of gauze that are depicted in FIGS. 5a, 5b and 5c. Thus, the closest-to-the-wound layer 80 (FIG. 5a) has a gauze web with a loose weave 84. The intermediate gauze layer 90 (FIG. 5b) has a tighter weave 92 and the upper layer 98 (FIG. 5c) has the most dense weave 100. These three layers, 80, 88, 96 are lightly adhered to each other at their respective edges 82, 90, 98 to form the composite bandage layer 70 shown in FIG. 5, with the idea that the lowest layer 80 has its gauze weave very sparsely done, while the middle layer 88 has an intermediary gauze pitch and the layer 96 having the densest weave. Therefore, the FIG. 5a gauze is closest to the wound and since its web lattice is the closest to the wound, as the wound heals, it might adhere to it but not as strongly as the denser weave in the layer 96 (FIG. 5c), making it less painful to remove the overall, composite bandage 70, by peeling away one layer at a time (with wetting being applied if needed).

Thereby, when removing the bandage 70, one would grasp the tabs 102, 94 and 86 one tab at a time, slowly peeling the bandage from the healing wound. Therefore, the removal of the dressing from the wounds (even wounds that have grown into the gauze) will cause less pain and less disturbance of the healing site.

Although the present invention has been described in relation to particular embodiments thereof, many other variations and modifications and other uses will become apparent to those skilled in the art. It is preferred, therefore, that the present invention be limited not by the specific disclosure herein, but only by the appended claims.

What is claimed is:

1. A multi-layer wound dressing, comprising:
a base layer comprising a circumscribing web of material defining a central opening of a pre-determined size to expose a wound area, the web having an adhering bottom surface for being adhered to and contact a region around the wound area;
an intermediate layer comprising a bandage layer, located over said opening of said base layer, the bandage layer being configured to be applied against the wound area; and
an outer, cover layer located over said bandage layer, said cover layer including a cover connection structure configured to make a sealed connection to said base layer, enabling repeated disconnection and reconnection of said cover layer to said base layer and repeated replacement of said bandage layer.

2. The multi-layer wound dressing of claim 1, wherein said cover connection structure comprises a tongue and groove connection.

3. The multi-layer wound dressing of claim 2, wherein said tongue and groove connection comprises a tongue structure on said cover layer and a circumscribing groove on said base layer.

4. The multi-layer wound dressing of claim 2, wherein said cover connection structure comprises an adhesive connection between said base layer and said cover layer.

5. The multi-layer wound dressing of claim 2, further including a bandage layer connection structure configured to connect an edge of said bandage layer to said base layer.

6. The multi-layer wound dressing of claim 5, wherein said cover connection structure is located more peripherally out than said bandage connection structure.

7. The multi-layer wound dressing of claim 1, wherein said bandage layer comprises gauze material.

8. The multi-layer wound dressing of claim 1, wherein an outer shape of said base layer is one of oblong, oval, square, rectangular and heart-shaped.

9. The multi-layer wound dressing of claim 1, further comprising orientation marks that enable the cover layer to be rotationally oriented relative to said base layer.

10. The multi-layer wound dressing of claim 9, wherein the orientation marks are provided on both the base layer and on the cover layer.

11. The multi-layer wound dressing of claim 1, further including cover layer tabs that are configured to be hand-graspable and constructed to allow lifting of the cover layer off the base layer.

12. The multi-layer wound dressing of claim 11, further comprising lifting tabs projecting from outer edge of the base layer for enabling grasping of the cover layer and removal of the same from a person's skin.

13. The multi-layer wound dressing of claim 1, wherein the bandage layer includes a central and removable wound-site bandage that is surrounded by a more rigid circumscribing material.

14. The multi-layer wound dressing of claim 1, wherein the bandage layer is formed of a plurality of overlapping webs of gauze material.

15. The multi-layer wound dressing of claim 14, wherein the bandage layer comprises at least three webs of gauze material that are peelable from each other.

16. The multi-layer wound dressing of claim 15, wherein the at least three layers include a lowest layer which comprises gauze material of a lowest weave density and a top-most layer that has a highest gauze weave density.

17. The multi-layer wound dressing of claim 1, wherein the cover layer is made from a semi-rigid material that resists bending, to prevent a force applied to it from the outside from being transmitted to the wound site.

18. The multi-layer wound dressing of claim 1, wherein the cover layer includes a transparent window.

19. The multi-layer wound dressing of claim 1, wherein the cover layer includes an outer cover with a plurality of perforations therein, said perforations having opening sizes small enough to substantially prevent outside moisture or wetness from entering the wound site and large enough to allow pressurized air or liquid underneath said cover layer to be vented to the atmosphere.
